(19) United States
(12) Patent Application Publication
(10) Pub. No.: US 2018/0236032 A1
(43) Pub. Date: Aug. 23, 2018

(54) COMBINATION COMPOSITION COMPRISING FGF-18 COMPOUND

(71) Applicant: MERCK PATENT GMBH, DARMSTADT (DE)

(72) Inventors: CHRISTOPH H. LADEL, DARMSTADT (DE); HANS GUEHRING, GEISENHEIM (DE); ANNE GIGOUT, GRIESHEIM (DE); CHRISTIAN BRENNEIS, KLEINOSTHEIM (DE)

(21) Appl. No.: 15/751,885

(22) PCT Filed: Aug. 11, 2016
(86) PCT No.: PCT/EP2016/069177

§ 371 Date: Feb. 12, 2018

(30) Foreign Application Priority Data
Aug. 13, 2015 (EP) 15180859.9

(51) Int. Cl. A61K 38/18; C07K 16/28; A61P 19/02

(52) U.S. Cl. CPC A61K 38/1825; C07K 16/2866; A61K 38/4893; A61K 2300/00; A61P 19/02

(57) ABSTRACT

The present invention relates to the use of an FGF-18 compound in combination with a further active ingredient, selected from the group of an inhibitor of IL-6, an inhibitor of IL-6 receptor, an inhibitor of NGF or a botulinum toxin compound. Said composition can be used for the treatment of a cartilage disorder such as osteoarthritis or cartilage injury.

### FIELD OF INVENTION

The present invention relates to the use of an FGF-18 compound in combination with a further active ingredient selected from the group consisting of an inhibitor of IL-6, an inhibitor of IL-6 receptor, an inhibitor of NGF or a botulinum toxin compound. Said composition can be used for the treatment of a cartilage disorder such as osteoarthritis or cartilage injury.

### BACKGROUND OF THE INVENTION

Cartilage is composed of chondrocytes (cells derived from mesenchymal cells) which are dispersed in the matrix (a firm, gel-like ground substance). The cartilaginous matrix is produced by these cells and comprises mainly Type II collagen fibres (except fibrocartilage which also contains type I collagen fibres), proteoglycans, and elastin fibres. Cartilage is found among other places in the joints, the rib cage, the ear, the nose, in the throat, in the trachea and in the intervertebral disks. There are three main types of cartilage: hyaline, elastic and fibrocartilage, providing different functional properties according to their histological morphology. Articular cartilage, for instance, is a hyaline cartilage, having viscoelastic properties, covering the articular surfaces of bones. The main purpose of articular cartilage is to provide smooth surfaces in order to ensure nearly frictionless movement of articulating bones.

Cartilage disorders broadly refer to diseases characterized by degeneration/disintegration of cartilage and abnormalities in the connective tissues which are manifested by inflammation, pain, stiffness and limitation of motion of the affected body parts. These disorders can be due to a pathology or can be the result of trauma or injury. Mature cartilage has very limited ability to self-repair, notably because mature chondrocytes have little potential for proliferation because of the limited supply with nutrients due to the absence of blood vessels in cartilage. Replacement of damaged cartilage, in particular articular cartilage, caused either by injury or disease is a major challenge for physicians, and available surgical treatment procedures are considered unpredictable and effective for only a limited time in younger patients without osteoarthritic changes. Therefore, the majority of patients either do not seek treatment or are counselled to postpone treatment for as long as possible. When treatment is required, the standard procedure is age dependent and varies between total or partly joint replacement, transplantation of pieces of cartilage or chondrocytes or marrow stimulating technique (such as microfracture). Microfracture is a cheap and common procedure that involves penetration of the subchondral bone to stimulate cartilage deposition by bone marrow derived stem cells. However, it has been shown that this technique does not repair sufficiently the chondral defect and the new cartilage formed is mainly fibrocartilage, resulting in a short-lived repair tissue. Indeed, fibrocartilage does not have the same biomechanical properties as hyaline articular cartilage and lacks often proper lateral integration into the surrounding cartilage. For this reason, the newly synthesized fibrocartilage may breakdown more easily (expected time frame: 5-10 years).

For patients with osteoarthritis all these cartilage repair techniques fail. The remaining non-surgical treatment consists notably of physical therapy, lifestyle modification (e.g. body weight reduction), supportive devices, oral drugs (e.g. non-steroidal anti-inflammatory drugs) and injection of drugs(e.g. hyaluronic acid and corticoids, and food supplementation. All these treatments are unable to stop OA disease progression. If the pain therapy also fails, surgery, such as joint replacement or high tibial osteotomy for the knee joint, are the remaining options for the patients. Tibial or femoral osteotomies (cutting the bone to rebalance joint wear) may reduce symptoms, help to maintain an active lifestyle, and delay the need for total joint replacement. Total joint replacement can provide relief for the symptom of advanced osteoarthritis, but generally requires a significant change in a patient's lifestyle and/or activity level.

Current available drug treatments are mainly directed to pain relief. At this time, there is no commercially available treatment that restores the cartilage damages (see Lotz, 2010). Interleukin 6 (IL-6) or Interleukin-6 receptor (IL-6R) are possible target to treat pain in osteoarthritis patient. It was indeed shown, in WO2005080429 for instance, that hind paw weight distribution (i.e. incapacitance test) was decreased when an IL-6 antibody was injected in the right arthritic knee of a mouse OA model, underlining the effect of an anti-IL-6 antibody on pain. Botulinum Toxin Type A has also been described in the context of pain linked to OA. There are more and more evidences to support its role in pain modulation (Boon et al., 2010). Pilot studies in humans have suggested efficacy in several different painful conditions, including pain related to spinal cord injury. Some preliminary data have been obtained for shoulder OA pain, with intra-articular injection of BoNT-A (Singh et al., 2009).

Anti-NGF compound is another category of compounds being described in the context of pain linked to OA. Currently, Tanezumab, Fasinumab or yet Fulranumab are being developed for treating pain in OA patients, and are all currently in phases II/III clinical trials for arthritis and/or chronic pain, based on promising results in phases I or II clinical trials (Sanga et al., 2013; Tiseo et al., 2014).

Fibroblast Growth factor 18 (FGF-18) is a member of the Fibroblast Growth Factor (FGF) family of proteins, closely related to FGF-8 and FGF-17. It has been shown that FGF-18 is a proliferative agent for chondrocytes and osteoblasts (Ellsworth et al., 2002; Shimoaka et al., 2002). FGF-18 has been proposed for the treatment of cartilage disorder such as osteoarthritis and cartilage injury either alone (WO2008023063) or in combination with hyaluronic acid (WO2004032849).

Various dosing regimen have been suggested for FGF-18. For instance, Moore et al. (2005) disclosed administration twice weekly for 3 weeks, and WO2008023063 taught administration once a week for 3 weeks. This last dosing regimen has been investigated in clinical trials.

Although the dosing regimen described in WO2008023063 gives good results in articular cartilage repair, there is a need of a method for decreasing pain/improving function, while maintaining the efficacy for the treatment of cartilage disorder. Indeed, pain is not only very often associated with cartilage disorders but represents the leading symptom for clinical detection of these disorders.

### SUMMARY OF THE INVENTION

It is an object of the present invention to provide the use of FGF-18 compound in combination with at least one further active ingredient, wherein said at least one further active ingredient is selected from the group consisting of an inhibitor of IL-6, an inhibitor of IL-6 receptor, an inhibitor of NGF or a botulinum toxin compound. The FGF-18 in combination with the further active ingredient can be used in the treatment of a cartilage disorder. Said cartilage disorder is for instance osteoarthritis or cartilage injury.

The present invention further provides a composition comprising a combination of at least two active ingredients, wherein one of the active ingredients is an FGF-18 compound and wherein the at least one other active ingredient is selected from the group consisting of an inhibitor of IL-6, an inhibitor of IL-6 receptor, an inhibitor of NGF or a botulinum toxin compound. In an embodiment, the composition of the at least two active ingredients is for use in the treatment of a cartilage disorder. Said cartilage disorder is for instance osteoarthritis or cartilage injury.

Also encompassed is an FGF-18 compound for use in the treatment of a cartilage disorder, in combination with at least one further active ingredient, wherein said at least one further active ingredient is selected from the group consisting of an inhibitor of IL-6, an inhibitor of IL-6 receptor, an inhibitor of NGF or a botulinum toxin compound. Said cartilage disorder is for instance osteoarthritis or cartilage injury.

Further provided is a kit comprising an FGF-18 compound together with instructions for simultaneous or sequential use with at least one further active ingredient, wherein said at least one further active ingredient is selected from the group consisting of an inhibitor of IL-6, an inhibitor of IL-6 receptor, an inhibitor of NGF or a botulinum toxin compound.

Also encompassed is a kit comprising an FGF-18 compound and at least one further active ingredient, wherein said further active ingredient is selected from the group consisting of an inhibitor of IL-6, an inhibitor of IL-6 receptor, an inhibitor of NGF or a botulinum toxin compound, together with instructions for use.

According to the invention as a whole, the FGF-18 compound and the at least one further active ingredient can be part of pharmaceutical formulations. The FGF-18 compound and at least one further active ingredient are part of a same pharmaceutical formulation or are each part of separate pharmaceutical formulations Said pharmaceutical formulations may further comprise at least one excipient.

Definitions

The term “FGF-18 compound” or “FGF-18”, as used herein, is intended to be a protein maintaining at least one biological activity of the human FGF-18 protein (i.e. Fibroblast Growth Factor 18). FGF-18 may be native, in its mature form, a recombinant form or a truncated form thereof. Biological activities of the human FGF-18 protein include notably the increase in chondrocyte or osteoblast proliferation (see WO9816644) or in cartilage formation (see WO2008023063). Native, or wild-type, human FGF-18 is a protein expressed by chondrocytes of articular cartilage. Human FGF-18 was first designated zFGF-5 and is fully described in WO9816644. SEQ ID NO:1 corresponds to the amino acid sequence of the native human FGF-18, with a signal peptide consisting of amino acid residues 1(Met) to 27(Ala). The mature form of human FGF-18 corresponds to the amino acid sequence from residue 28(Glu) to residue 207(Ala) of SEQ ID NO: 1 (180 amino acids).

FGF-18, in the present invention, may be produced by recombinant method, such as taught by the application WO2006063362. Depending on the expression systems and conditions, FGF-18 in the present invention is expressed in a recombinant host cell with a starting Methionine (Met) residue or with a signal sequence for secretion. When expressed in prokaryotic host, such as in E. coli, FGF-18 contains an additional Met residue in N-terminal of its sequence. For instance, the amino acid sequence of human FGF-18, when expressed in E.coli, starts with a Met residue in N-term (position 1) followed by residues 28 (Glu) to residue 207 (Ala) of SEQ ID NO: 1.

The term “truncated form” of FGF-18, as used herein, refers to a protein which comprises or consists of residues 28(Glu) to 196(Lys) of SEQ ID NO: 1. Preferably, the truncated form of FGF-18 protein is the polypeptide designated “trFGF-18” (170 amino acids; also known as rhFGF-18 or sprifermin), which starts with a Met residue (in N-terminal) followed by amino acid residues 28 (Glu) -196 (Lys) of the wild-type human FGF-18. The amino acid sequence of trFGF-18 is shown in SEQ ID NO:2 (amino acid residues 2 to 170 of SEQ ID NO:2 correspond to amino acid residues 28 to 196 of SEQ ID NO:1). trFGF-18 is a recombinant truncated form of human FGF-18, produced in E.coli (see WO2006063362). trFGF-18 has been shown to display similar activities as the mature human FGF-18, e.g. it increases chondrocyte proliferation and cartilage deposition leading to repair and reconstruction for a variety of cartilaginous tissues (see WO2008023063).

The term “inhibitor of IL-6” as used herein refers to a compound that is able to inhibit the activity of IL-6 (i.e. Interleukin 6), either partly or completely. The preferred “inhibitor of IL-6” according to this invention is an antibody, or fragments thereof, as well as a nanobody. Such a compound is for instance, but not limited to, siltuximab (See SEQ ID Nos. 4-5) or PMP6B6 (See SEQ ID No. 6). Dazakinumab, clazakizumab, Sirukumab, Olokizumab or OP-R003 are other examples of known IL-6 inhibitors (specific sequences not known).

The term “inhibitor of IL-6 receptor” as used herein refers to a compound that is able to inhibit the activity of IL-6 receptor (i.e. Interleukin 6 Receptor), either partly or completely. The preferred “inhibitors of IL-6 receptor” according to this invention is an antibody, or fragments thereof, as well as a nanobody. Such a compound is for instance, but not limited to, tocilizumab (See SEQ ID Nos. 7-8). SA-237 or ALX-0061 are other examples of known IL-6 receptor inhibitors (specific sequences not known).

The term “inhibitor of NGF” as used herein refers to a compound that is able to inhibit the activity of NGF (i.e. Nerve Growth Factor), either partly or completely. The preferred “inhibitors of NGF” according to this invention is an antibody, or fragments thereof, as well as a nanobody. Such a compound is for instance, but not limited to, Tanezumab (See SEQ ID Nos. 9-10), Fasinumab (See SEQ ID Nos. 11-12), Fulranumab (See SEQ ID Nos. 13-14). ANA-02, ABT-110, ALD-906 or MEDI-578 are other examples of known NGF receptor inhibitors (specific sequences not known).

The term “botulinum toxin compound” as used herein refers to a neurotoxic protein produced by the bacterium Clostridium botulinum and related species. The preferred “botulinum toxin compound” to be used according to this invention is the botulinum toxin type A (also known as BoNT-A or BoNT/A; see SEQ ID No. 3). Such compounds are for instance the compounds known by as abobotulinumtoxinA, OnabotulinumtoxinA, incobotulinumtoxinA.

The term “treatment cycle” or “cycle” corresponds to the period wherein an FGF-18 compound in combination with at least one further active ingredient. For instance, one cycle can consist of 3 injections of an FGF-18 compound in combination with at least one further active ingredient, once per week. Such a “treatment cycle” can be repeated. For instance, a second “treatment cycle” can be performed 3, 4, 5 or 6 months after the last injection of the previous cycle. Alternatively, a second cycle can also be performed 1 year or 2 years after the first injection in the first cycle.

The term “cartilage disorder”, as used herein, encompasses disorders resulting from damages due to injury, such as traumatic injury, chondropathy or arthritis. Examples of cartilage disorders that may be treated by the administration of the FGF-18 formulation described herein include but are not restricted to arthritis, such as osteoarthritis, and cartilage injury. Degenerative diseases/disorders of the cartilage or of the joint, such as chondrocalcinosis, polychondritis, relapsing polychondritis, ankylosing spondylitis or costochondritis are also encompassed by this wording. The International Cartilage Repair Society has proposed an arthroscopic grading system to assess the severity of the cartilage defect: grade 0: (normal) healthy cartilage, grade 1: the cartilage has a soft spot or blisters, grade 2: minor tears visible in the cartilage, grade 3: lesions have deep crevices (more than 50% of cartilage layer) and grade 4: the cartilage tear exposes the underlying (subchronal) bone. (see ICRS publication: http://www.cartilage.orq/_files/contentmanagement/ICRS_evaluation.pdf, page 13).

The term “arthritis” as used herein encompasses disorders such as osteoarthritis, rheumatoid arthritis, juvenile rheumatoid arthritis, infectious arthritis, psoriatic arthritis, Still's disease (onset of juvenile rheumatoid arthritis) or osteochondritis dissecan. It preferably includes diseases or disorders in which ones the cartilage is damaged.

The term “Osteoarthritis” is used to intend the most common form of arthritis. The term “osteoarthritis” encompasses both primary osteoarthritis and secondary osteoarthritis (see for instance The Merck Manual, 17th edition, page 449). Osteoarthritis may be caused by the breakdown of cartilage. Bits of cartilage may break off and cause pain and swelling in the joint between bones. Over time, the cartilage may wear away entirely, and the bones will rub together. Osteoarthritis can affect any joint but usually concerns hands, shoulders and weight-bearing joints such as hips, knees, feet, and spine. In a preferred example, the osteoarthritis may be knee osteoarthritis or hip osteoarthritis. This wording encompasses notably the forms of osteoarthritis which are classified as stage 1 to stage 4 or grade 1 to grade 6 according to the OARSI classification system. The skilled person is fully aware of osteoarthritis classifications that are used in the art, in particular said OARSI assessment system (also named OOCHAS; see for instance Custers et al., 2007). Osteoarthritis is one of the preferred cartilage disorders that can be treated by administering the FGF-18 compounds according to the present invention.

The term “cartilage injury” as used herein is a cartilage disorder or cartilage damage resulting notably from a trauma. Cartilage injuries can occur notably after traumatic mechanical destruction, notably further to an accident or surgery (for instance microfracture surgery). This term “cartilage injury” also includes chondral or osteochondral fracture and damage to meniscus. Also considered within this definition is sport-related injury or sport- related wear of tissues of the joint. The term also includes microdamage or blunt trauma, a chondral fracture, an osteochondral fracture or damage to meniscus.

### DETAILED DESCRIPTION OF THE INVENTION

It has surprisingly been found that the compositions of and uses according to the present invention at least maintain the activities of sprifermin. Indeed, it was found that in overall 1) the effects of an FGF-18 compound are not impacted by an inhibitor of IL-6, an inhibitor of IL-6 receptor, an inhibitor of NGF or a botulinum toxin compound when administered according to the compositions and uses disclosed herein and 2) that an FGF-18 compound does not affect the effect of an inhibitor of IL-6, an inhibitor of IL-6 receptor, an inhibitor of NGF or a botulinum toxin compound when administered according to the compositions and uses disclosed herein. This finding was not expected because of the high molecular weight of each compound of the combination. Also surprising, said activities are maintained, even at very low dosage for each compound. Not only the combinations in overall maintain the respective activities, but further surprisingly, the anabolic effects of FGF-18 can be potentiated (see examples 1 and 2 for instance). Another advantage of the present invention is that it will allow to decrease pain/improve function, while at least maintaining the efficacy of FGF-18 for the treatment of cartilage disorder.

The present invention provides the use of FGF-18 compound in combination with at least one further active ingredient (herein indifferently alternatively called “additional active ingredient” or “other active ingredient”), wherein said at least one further active ingredient is selected from the group consisting of an inhibitor of IL-6, an inhibitor of IL-6 receptor, an inhibitor of NGF or a botulinum toxin compound. In an embodiment, the FGF-18 in combination with the at least one further active ingredient are for use in the treatment of a cartilage disorder. Said cartilage disorder is for instance osteoarthritis or cartilage injury.

In a further particular embodiment, the FGF-18 compound in combination with the at least one further active ingredient are administered intra-articularly. Alternatively, the FGF-18 compound is administered intra-articularly and the at least one further active ingredient is administered intravenously or subcutaneously.

The FGF-18 compound can be administered in combination with the at least one further active ingredient, either simultaneously (co-administration), or sequentially (in any order). Should the FGF-18 compound and the at least one further active ingredient being administered sequentially, said sequential administration will be preferably done during the same visit to the doctor.

Also encompassed by the invention is an FGF-18 compound for use in the treatment of a cartilage disorder.in combination with at least one further active ingredient, wherein said at least one further active ingredient is selected from the group consisting of an inhibitor of IL-6, an inhibitor of IL-6 receptor, an inhibitor of NGF or a botulinum toxin compound. Said cartilage disorder is for instance osteoarthritis or cartilage injury. The FGF-18 compound in combination with the further active ingredient are preferably administered intra-articularly. Alternatively, the FGF-18 compound is administered intra-articularly and the at least further active ingredient is administered intravenously or subcutaneously.

The FGF-18 compound can be administered in combination with the at least one further active ingredient, either simultaneously (co-administration), or sequentially (in any order). Should the compounds being administered sequentially, said sequential administration will be preferably done during the same visit to the doctor.

The present invention further provides a composition comprising a combination of at least two active ingredients, wherein one of the active ingredients is an FGF-18 compound and wherein the at least one other active ingredient is selected from the group consisting of an inhibitor of IL-6, an inhibitor of IL-6 receptor, an inhibitor of NGF or a botulinum toxin compound.

In an embodiment, the composition of the at least two active ingredients is for use in the treatment of a cartilage disorder. Said cartilage disorder is for instance osteoarthritis or cartilage injury.

In a further particular embodiment, the composition of the at least two active ingredients is administered intra-articularly.

In the context of the invention, the composition comprising a combination of the at least two active ingredients further comprises at least one excipient. The at least one excipient is for instance a buffer, a surfactant, a salt, an antioxidant, a isotonicity agent, a bulking agent, a stabilizer or any combination thereof.

Further provided is a kit comprising an FGF-18 compound together with instructions for simultaneous or sequential use (in any order) in combination with at least one further active ingredient, wherein said at least one further active ingredient is selected from the group consisting of an inhibitor of IL-6, an inhibitor of IL-6 receptor, an inhibitor of NGF or a botulinum toxin compound. The FGF-18 compound and the at least one further active ingredient can each be part of a separate pharmaceutical formulation. In such a case, each pharmaceutical formulation can further comprise at least one pharmaceutically acceptable carrier, excipients or the like.

Also encompassed is a kit comprising an FGF-18 compound and at least one other active ingredient, wherein said at least one other active ingredient is selected from the group consisting of an inhibitor of IL-6, an inhibitor of IL-6 receptor, an inhibitor of NGF or a botulinum toxin compound, together with instructions for use. The FGF-18 compound and the other active ingredient can be part of the same pharmaceutical formulation or each part of a separate pharmaceutical formulation. Said pharmaceutical formulation(s) can further comprise at least one pharmaceutically acceptable carrier, excipients or the like.

The FGF-18 compound of the invention as a whole is preferably selected from the group consisting of a) a polypeptide comprising or consisting of the human FGF-18 mature form comprising residues 28-207 of SEQ ID NO:1, or b) a polypeptide comprising or consisting of FGF-18(170AA)(SEQ ID NO.2). Particularly, this compound is selected from human wildtype mature FGF-18 or trFGF-18. Said compound increases cartilage deposition and allows cartilage repair. The FGF-18 compound is preferably administered intra-articularly at a dose of 3-600 micrograms (pg or mcg), preferably 3-300 pg, or preferably 10-200 μg, or more preferably 30-150 μg, or even more preferably 30-120 μg per single administration. In a preferred embodiment the treatment comprises administration at a dose of or of about 3, 10, 20, 30, 40, 50, 60, 90, 100, 120, 150, 180, 200, 240 or 300 μg per single intra-articular administration of the FGF-18 compound. Preferred doses include 10, 20, 30, 60, 90, 120, 180, 240 or 300 μg per single intra-articular administration of the FGF-18 compound. It should be understood that the dose of the FGF-18 compound to be administered will be different should the patient to be treated be a human or a non-human mammal. For instance, for dogs, the dose will be preferably 5-fold less important than for human. As an example, should the human dose be range from 30 to 120 μg per single intra-articular administration, the dose for a dog could be ranged from 5 to 20 μg per single intra-articular administration.

In the context of the present invention as a whole, the IL-6 inhibitor is preferably an antibody against IL-6 (alternatively named anti-IL-6 antibody) or a nanobody targeting IL-6 (alternatively named anti-IL-6 nanobody). Examples of such inhibitors are found in the definitions section. Said IL-6 inhibitor can be administered ata dose of 0.001 - 1000 milligrams (mg), preferably 0.1-500 mg, or more preferably 0.2-250 mg per single administration. In a preferred embodiment the treatment comprises administration at a dose of about 0.01, 0.02, 0.03, 0.1, 0.2, 0.3, 0.5, 1, 1.5, 2, 5, 10, 15, 20, 30, 40, 50, 60, 70, 80, 90, 100, 150, 200, 250 or 300 mg per single administration of the IL-6 inhibitor. Alternatively, the known dosing regimen for a given drug can be used. It should be understood that the dose of IL-6 inhibitor will be different should the patient to be treated be a human or a non-human mammal. For instance, for dogs, the dose will be preferably 6-fold less important than for human. As an example, should the human dose of IL-6 inhibitor be 2 mg per single administration, the dose for a dog could be about 0.35 mg per single administration. The doctor will adapt the dosing regimen for the IL-6 inhibitor case by case, depending on the patient.

In the context of the present invention as a whole, the IL-6 receptor inhibitor is preferably an antibody against IL-6 receptor (alternatively named anti-IL-6R antibody) or a nanobody targeting IL-6 receptor (alternatively named anti-IL-6R nanobody). Examples of such inhibitors are found in the definitions section. Said IL-6 receptor inhibitor can be administered at a dose of 0.001 - 500 milligrams (mg), preferably 0.1-250 mg, or more preferably 0.5-200 mg per single administration. In a preferred embodiment the treatment comprises administration at a dose of about 0.01, 0.03, 0.1, 0.25, 0.3, 0.5, 1, 1.5, 2, 5, 10, 15, 20, 30, 40, 50, 60, 70, 80, 90, 100, 150, 200, 250 or 300 mg per single administration of the IL-6R inhibitor. Alternatively, the known dosing regimen for a given drug can be used. Tocilizumab for instance is approved in the treatment of rheumatoid arthritis at a dosing of 4 mg per kilogram, when administered intravenously, or at 162 mg, when administered subcutaneously. It should be understood that the dose of IL-6R inhibitor will be different should the patient to be treated be a human or a non-human mammal. For instance, for dogs, the dose will be preferably 6-fold less important than for human. As an example, should the human dose of IL-6R inhibitor be 150 mg per single administration, the dose for a dog could be 25 mg per single administration. The doctor will adapt the dosing regimen for the IL-6R inhibitor case by case, depending on the patient. In the context of the present invention as a whole, the NGF inhibitor is preferably an antibody against NGF (alternatively named anti-NGF antibody) or a nanobody targeting NGF (alternatively named anti-NGF nanobody). Examples of such inhibitors are found in the definitions section. Said NGF inhibitor can be administered at a dose of 0.01-250 milligrams (mg), preferably 0.1-100 mg, or more preferably 0.5-75 mg per single administration. In a preferred embodiment the treatment comprises administration ata dose of about 0.03, 0.1, 0.25, 0.3, 0.5, 1, 1.5, 2, 3, 5, 10, 15, 20, 30, 40, 50, 60, 70, 80, 90, 100 or 150 mg per single administration of the NGF inhibitor. Alternatively, the known dosing regimen for a given drug can be used. It should be understood that the dose of NGF inhibitor will be different should the patient to be treated be a human or a non-human mammal. For instance, for dogs, the dose will be preferably 6-fold less important than for human. As an example, should the human dose of NGF inhibitor be 10 mg per single administration, the dose for a dog could be about 1.5 mg per single administration. The doctor will adapt the dosing regimen for the NGF inhibitor case by case, depending on the patient.

In the context of the present invention as a whole, the botulinum toxin compound, preferably the botulinum toxin type A (see definition section) can be administered ata dose of 0.1-1000 Units (U), preferably 0.2-500 U, or more preferably 0.5-300 U per single administration. In a preferred embodiment the treatment comprises administration at a dose of about 0.3, 0.5, 1, 5, 10, 15, 20, 30, 50, 100, 125, 150, 175, 200, 250 or 300 U per single administration of the botulinum toxin compound. Alternatively, the known dosing regimen for a given drug can be used. It should be understood that the dose of botulinum toxin compound will be different should the patient to be treated be a human or a non-human mammal. For instance, for dogs, the dose will be preferably 6-fold less important than for human. As an example, should the human dose of botulinum toxin compound be 100 U per single administration, the dose for a dog could be about 15 U per single intra-articular administration. The doctor will adapt the dosing regimen for the botulinum toxin compound case by case, depending on the patient.

In the context of the invention as a whole, the FGF-18 compound and the at least one further active ingredient are part of pharmaceutical formulations. The FGF-18 compounds and/or the at least one other active ingredient may be formulated as pharmaceutical composition(s), i.e. together with at least one pharmaceutically acceptable carrier, excipients or the like. The definition of “pharmaceutically acceptable” is meant to encompass any carrier, excipients or the like, which does not interfere with effectiveness of the biological activity of the active ingredient and that is not toxic to the patient to which it is administered. The at least one excipient is for instance selected from the group consisting of a buffer, a surfactant, a salt, an antioxidant, a isotonicity agent, a bulking agent, a stabilizer or any combination thereof. For example, for parenteral administration, the active protein(s) may be formulated in a unit dosage form for injection in vehicles such as saline, dextrose solution, serum albumin and Ringers solution. Formulations for intraarticular application will comply with most of the requirements that also apply to other injection formulations, i.e., they need to be sterile and compatible with the physiological conditions at the application site (e.g., knee joint, synovial fluid). The excipients used for intraarticular injection may also be present in other injection formulations, e.g., for intravenous or subcutaneous application. Such formulations of FGF-18 compounds and/or at least one further active ingredient, including at least one further pharmaceutically acceptable carrier, excipients or the like, are also useful in the context of the present invention.

In the context of the invention as a whole, the FGF-18 compound in combination with the at least one other active ingredient will be useful for treating cartilage disorders, such as osteoarthritis or cartilage injury. In particular it can be used for treating articular cartilage defects in synovial joints that are, for instance, due to superficial fibrillation (early osteoarthritis), cartilage degeneration due to osteoarthritis, and chondral or osteochondral defects due to injury or disease. FGF-18 compounds in combination with the at least one further active ingredient may also be used for treating joint disease caused by osteochondritis dissecans and degenerative joint diseases. In the field of reconstructive and plastic surgery, FGF-18 compounds in combination with the at least one other active ingredient will be useful for autogenous or allogenic cartilage expansion and transfer for reconstruction of extensive tissue defects. FGF-18 compositions can be used to repair cartilage damage in conjunction with lavage of the joint, stimulation of bone marrow, abrasion arthroplasty, subchondral drilling, or microfracture of the subchondral bone.

In a preferred embodiment, the cartilage disorder to be treated according to the invention is osteoarthritis, such as knee osteoarthritis or hip osteoarthritis. The osteoarthritis to be treated can be, for example, and not limited to, primary osteoarthritis or secondary osteoarthritis, as well as osteoarthritis which is classified as stage 1 to stage 4 or grade 1 to grade 6 according to the OARSI classification system.

In another preferred embodiment, the cartilage disorder to be treated according to the invention is cartilage injury with and without surgical interventions as microfractures. Additionally, after the growth of cartilage due to the administration of the FGF-18 compound in combination with the at least a further active ingredient, a surgical treatment may be necessary to suitably contour the newly formed cartilage surface.

In a preferred embodiment, the treatment comprises peri-synovial administration, intra-synovial administration, peri-articular administration or intra-articular administration of the FGF-18 compound, either alone or together with the at least one other active ingredient. FGF-18 compounds can be applied, either alone or together with the at least one other active ingredient, by direct injection into the synovial fluid of the joint or directly into the defect, either alone or complexed with a suitable carrier for extended release of protein (e.g. sustained-release formulations) or restricted local release. Should the at least one other active ingredient not being administered according to the same administration mode as the FGF-18 compound, it can be administered intravenously or subcutaneously. The intraarticular administration is done in a joint selected from joint of the hip, knee, elbow, wrist, ankle, spine, feet, finger, toe, hand, shoulder, ribs, shoulder blades, thighs, shins, heels and along the bony points of the spine. In yet another preferred embodiment the intraarticular administration is done in a the joint of the hip or the knee.

For the treatment of the cartilage disorder, the FGF-18 compound in combination with the at least one further active ingredient can be administered for at least one treatment cycle. A treatment cycle can consist, as an example, of three injections of an FGF-18 compound in combination with at least one further active ingredient, once per week. Such a treatment cycle can be repeated. For instance, a second treatment cycle can be performed 3, 4, 5 or 6 months after the last injection of the previous cycle. Alternatively, a second cycle can also be performed 1 year or 2 years after the first injection in the first cycle.

### DESCRIPTION OF THE FIGURES:

FIG. 1: BaF3/FGFR3 cells were cultured 48 h with CNTO328 or PMP6B6 and with Sprifermin (squares) or without Sprifermin (circles). CTR+ is the O.D. obtained with cells cultured with Sprifermin only and CTR− with cells cultured without Sprifermin. Cells cultured with CNTO328 or PMP6B6 and Sprifermin were compared to CTR+ while cells cultured without Sprifermin were compared with CTR−. Symbols represent the average +/−SEM. “*” means “different” with p<0.05

FIG. 2: Human chondrocytes cultured seven days in presence of CNTO328 or PMP6B6 in presence (squares) or in absence (circles) of Sprifermin. The cell density and the GAG production were evaluated. Symbols represent the average +/−SEM. “*” means “different” with p<0.05 from the same CNTO328 or PMP6B6 concentration but without FGF-18. “#” means “different” with p<0.05 from the control without CNTO328 or PMP6B6 (0 ng/mL).

FIG. 3: Human chondrocytes cultured seven days in presence of CNTO328 or PMP6B6 in presence (squares) or in absence (circles) of Sprifermin. The expression of Collagen type I, II, Sox9 were evaluated. Symbols represent the average +/−SEM. −. “*” means different with p<0.05 from the same CNTO328 or PMP6B6 concentration but without FGF-18. “#” means different with p<0.05 from the control without CNTO328 or PMP6B6 (0 ng/mL).

FIG. 4: BaF3/FGFR3 cells were cultured 48 h with Actemra and with Sprifermin (squares) or without Sprifermin (circles). CTR+ is the O.D. obtained with cells cultured with Sprifermin 100 ng/mL only and CTR− with cells cultured without Sprifermin. Cells cultured with Actemra and Sprifermin were compared to CTR+ while cells cultured without Sprifermin were compared with CTR−. Symbols represent the average +/−SEM. “*” means different with p<0.05.

FIG. 5: Human chondrocytes cultured seven days in presence of Actemra in presence (squares) or in absence (circles) of Sprifermin. The cell density and the GAG production were evaluated. Symbols represent the average +/−SEM. “*” means different with p<0.05 from the same Actemra concentration but without FGF-18. “#” means different with p<0.05 from the control without Actemra (0 ng/mL).

FIG. 6: Human chondrocytes cultured seven days in presence of Actemra in presence (squares) or in absence (circles) of Sprifermin. The expression of Collagen type I, II, Sox9 were evaluated. Symbols represent the average +/−SEM. −. “*” means different with p<0.05 from the same Actemra concentration but without FGF-18. “#” means different with p<0.05 from the control without Actemra (0 ng/mL).

FIG. 7: BaF3/FGFR3 cells were cultured 48 h with Tanezumab and with Sprifermin (squares) or without Sprifermin (circles). CTR+is the O.D. obtained with cells cultured with Sprifermin 100 ng/mL only. Cells cultured with Tanezumab and Sprifermin were compared to CTR+. Symbols represent the average +/−SEM. “*” means different with p<0.05.

FIG. 8: BaF3/FGFR3 cells were cultured 48 h with Xeomin® and with (square) Sprifermin or without (circles) Sprifermin. CTR+ is the O.D. obtained with cells cultured with Sprifermin only and CTR− with cells cultured without any compound. Cells cultured with Xeomin® and Sprifermin were compared to CTR+ while cells cultured without Sprifermin were compared with CTR−. “*” means different with p<0.01.

FIG. 9: Bovine chondrocytes cultured seven days in presence of Xeomin® in presence (squares) or in absence (circles) of Sprifermin. The cell density and the GAG production were evaluated. Cells cultured with Xeomin® were compared to their respective controls (0 mU/mL Xeomin, with or without Sprifermin). Symbols represent the mean +/−SEM. “*” means different with p<0.01.

FIG. 10: Bovine chondrocytes cultured seven days in presence of Xeomin® in presence (squares) or in absence (circles) of Sprifermin. The expression of Collagen type I, II, Sox9 and aggrecan were evaluated Cells cultured with Xeomin® were compared to their respective controls (0 mU/mL Xeomin, with or without Sprifermin). Symbols represent the mean +/- SEM. “*” means different with p<0.01.

### DESCRIPTION OF THE SEQUENCES:

SEQ ID NO.1: Amino acid sequence of the native human FGF-18.

SEQ ID NO.2: Amino acid sequence of the recombinant truncated FGF-18 (trFGF-18).

SEQ ID NO.3: Amino acid sequence of Botulinum Neurotoxin Type A (Xeomin®)

SEQ ID NO.4: Amino acid sequence of heavy chain of CNTO328 (siltuximab)

SEQ ID NO.5: Amino acid sequence of light chain of CNTO328 (siltuximab)

SEQ ID NO.6: Amino acid sequence of PMP6B6

SEQ ID NO.7: Amino acid sequence of heavy chain of tocilizumab (Actemra®)

SEQ ID NO.8: Amino acid sequence of light chain of tocilizumab (Actemra®)

SEQ ID NO.9: Amino acid sequence of heavy chain of tanezumab

SEQ ID NO.10: Amino acid sequence of light chain of tanezumab

SEQ ID NO.11: Amino acid sequence of heavy chain of Fasinumab

SEQ ID NO.12: Amino acid sequence of light chain of Fasinumab

SEQ ID NO.13: Amino acid sequence of heavy chain of Fulranumab

SEQ ID NO.14: Amino acid sequence of light chain of Fulranumab

### EXAMPLES

Material

FGF-18 compound: The recombinant truncated FGF-18 (trFGF-18) of the present examples has been prepared by expression in E.coli , according to the technique described in the application WO2006063362. In the following examples, trFGF-18 and FGF-18 are used interchangeably. It was formulated in 7 mM Na2HPO4, 1 mM KH2PO4, 2.7 mM KCl, pH 7.3.

Botulinum toxin compound: The Botulinum Neurotoxin Type A of the present examples is Xeomin® (Merz, Frankfurt, Germany). It was formulated in 4,7 mg/mL Sucrose, 1 mg/mL HAS.

IL-6 inhibitors: The IL-6 inhibitors of the present examples are:
CNTO328 (Siltuximab) is an anti-IL-6 antibody. It was formulated in PBS.
PMP6B6 is a nanobody targeting IL-6. It was formulated in BMM2.

IL-6 receptor inhibitor: The IL-6 receptor inhibitor of the present examples is tocilizumab (Actemra®). NGF inhibitor: the NGF inhibitor of the present examples is tanezumab.

### Example 1

### Combination of FGF-18 and Inhibitors of IL-6

Methods:

BaF3/FGFR3c bioassay: The day before the assay starts, 1×10⁷ cells were seeded in 20 mL of assay medium in a 75 cm² flask for 24 hours at 37° C., 5% CO₂ for a IL-3 starvation step. At the day of the assay 20 000 cells/well were seeded in a 96 well plate in 50 μL of assay medium containing either CNTO328 at 0.1, 1, 10, 100, 1 000 and 10 000 ng/mL or PMP6B6 at 0.001, 0.01, 0.1, 1, 10 and 100 ng/mL and containing Sprifermin 100 ng/mL or not. As controls, cells were also cultivated with Sprifermin 100 ng/mL alone (positive control, CTR+ on the graph), with BMM2 1/2200, or without any compound (both negative controls, CTR− on the graph). All conditions were realized with N=6. Cells were cultivated 2 days at 37° C., 5% CO₂, and the metabolic activity was measured with the WST-1 reagent (Roche).

Primary human chondrocyte culture: After cell isolation human chondrocytes were inoculated at 14-18 million cells in a 75 cm² flask and cultured for seven to twelve days in complete HAM's F12. Cells were then harvested with accutase and counted before being inoculated in a 24-well plate at 200000 cells/well in one mL of complete HAM's F12 supplemented with different concentrations of either CNTO328 (1, 10, 100, 1 000 ng/mL) or PMP6B6 (0.1, 1, 10 and 100 ng/mL) in presence or absence of Sprifermin 100 ng/mL. As controls, cells were also cultivated with Sprifermin 100 ng/mL alone (positive control), with BMM2 1/2200 or without any compound (both negative controls). The results for the negative controls are shown at the values 0 ng/mL for the PMP6B6 and CNTO328 concentrations. All conditions were realized with N=3. Cells were cultivated seven days at 37° C., 5% CO₂, and a complete medium change was performed after three days. At the end of the culture, the cells were detached with accutase (Sigma-Aldrich) and the cell concentration evaluated with a Vicell. (Beckmann Coulter).

The dimethylmethylene blue (DMMB) assay was used to quantify glycosaminoglycan (GAG) in the culture media harvested after seven days of culture. 50 μL of the samples were mixed with 200 μL of DMMB reagent (16mg/mL DMMB in ethanol, formic acid and nitrogen formate) in a 96 well plates. The absorbance at 525 nm was read and compared to that of chondroitin sulfate C standards (Sigma Aldrich). The GAG concentration (μg/mL) was divided by the cell concentration (million cells/mL) to normalize the GAG production in (μg/million cells)

Gene expression was analysed by quantitative PCR. RNA was first isolated with a RNeasy minikit, (Qiagen) and cDNA synthesized with the SuperScript III First-Strand Synthesis SuperMix (Sigma-Aldrich). The cDNA was then digested by RNAse H to digest RNA and analysed by qPCR with the SYBRGreen Jumpstart Taq Ready Mix in presence of reverse and forward primers at 200 nM in the thermocycler Mx3000P (Agilent Technologies).

Results:

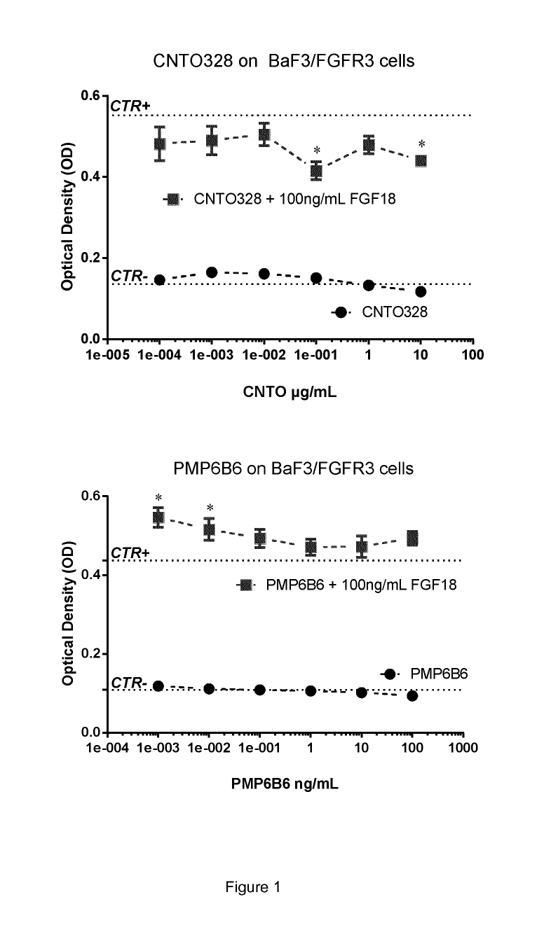

BaF/FGFR3 cell assay (FIG. 1): In the absence of Sprifermin, the increasing concentrations of CNTO328 or PMP6B6 did not influence the cell proliferation and the O.D. remained low. As expected, the BaF3/FGFR3 cell proliferation increased in presence of Sprifermin, resulting in an Optical Density (O.D.) increasing from about 0.12 (CTR−) to about 0.5 (CTR+). In the presence of Sprifermin, CNTO328 and PMP6B6 did not show any clear trend. Some fluctuations of the O.D. were observed but it stayed in the range of the O.D. observed with Sprifermin alone. Consequently, it can be concluded that neither CNTO328 nor PMP6B6 negatively influenced the effect of Sprifermin on BaF3/FGFR3 cells.

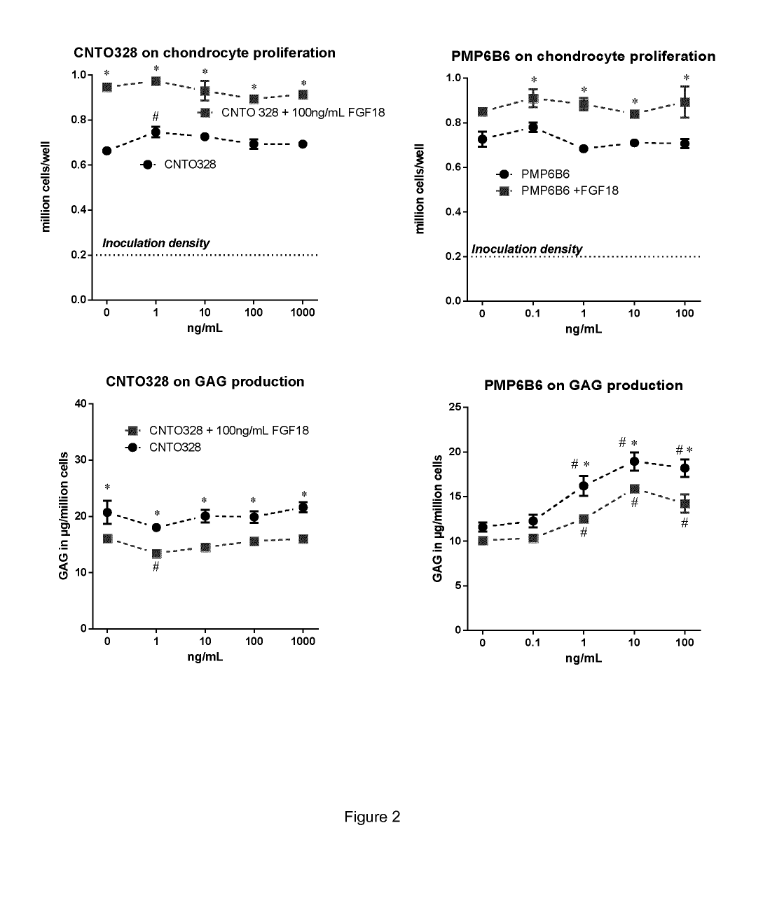

Human chondrocytes—Proliferation and GAG production (FIG. 2): Sprifermin increased chondrocyte proliferation, resulting at the end of the culture in a higher cell concentration (about 0.7 million cells/well in absence of Sprifermin and about 0.9 million cells/well in presence of Sprifermin). This effect was maintained in presence of CNTO328 and PMP6B6. Similarly, no effect of both anti-IL-6 on proliferation could be observed in absence of Sprifermin. The GAG production was slightly decreased when the cells were cultured in continuous presence of Sprifermin. Both in absence or presence of Sprifermin, CNTO328 was found to have no effect on GAG production. On the contrary, PMP6B6 was found to increase GAG production dose dependently in both presence and absence of Sprifermin.

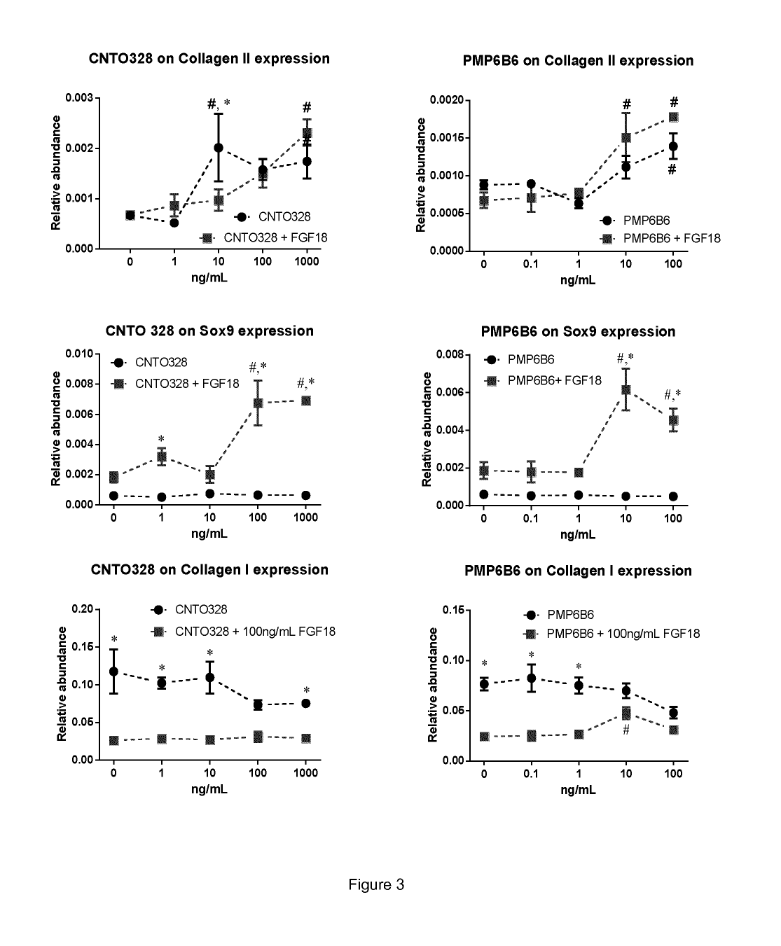

Human chondrocytes—Gene expression (FIG. 3): in human OA chondrocytes cultured in monolayer, Sprifermin down-regulated Collagen I expression (from 0.12 to 0.025) while increasing Sox9 expression (from 0.00060 to 0.0018) and had no effect on Collagen II expression.

Both CNTO328 and PMP6B6 were found to increase Collagen type II expression in a dose-dependent way. With CNTO328 1 000 ng/mL Collagen type II expression was increased by 2.5 fold in absence of Sprifermin and surprisingly by 2.9 fold in presence of Sprifermin. In presence of PMP6B6 100 ng/mL, Collagen II expression increased by 1.6 and more surprisingly by 2.6 fold in absence or presence of Sprifermin respectively.

Similarly CNTO328 and PMP6B6 increased Sox9 expression but only in presence of Sprifermin. The expression was surprisingly increased by 3.85 and 2.5 fold in presence of CNTO328 (100-1000 ng/mL or PMP6B6 (10-100 ng/mL) respectively for chondrocytes cultured with Sprifermin. Collagen I expression was mostly unchanged by CNTO328 and PMP6B6 in presence of Sprifermin, compared to Sprifermin alone. In absence of Sprifermin however and with increasing concentrations of CNTO328 and PMP6B6, Collagen I expression decreased.

Conclusions:

As a conclusion anti-IL-6 antibodies or fragments thereof, such as CNTO328 and PMP6B6, do not interfere with FGF-18. Inhibitors of IL-6 also showed a clear, dose-dependent anabolic effect on human OA chondrocytes, in particular when combined with FGF-18. Surprisingly, the combinations of FGF-18 with IL-6 inhibitors have a synergistic effect on Sox9 expression, which is known to be required for cartilage formation and for expression of chondrocyte-specific genes. This surprising effect could be due to a reduction of the inflammatory environment by the anti-IL-6 compounds, thus potentiating the FGF-18 effect on Sox9 expression. In overall, IL-6 inhibitors are able to increase the anabolic effect of FGF-18.

### Example 2

### Combination of FGF-18 and Inhibitors of IL-6 Receptor

Methods: BaF3/FGFR3c bioassay: The same method and conditions as the one described in example 1 were used. At the day of the assay 20 000 cells/well were seeded in a 96 well plate in 50 μL of assay medium containing tocilizumab (from Roche) at 0.001, 0.01, 0.1, 1, 10 or 100 μg/mL and containing Sprifermin 100 ng/mL or not. The controls were realised with cells cultured with (CTR +) or without Sprifermin (CTR-) and in presence of the excipients of the Tocilizumab formulation (15 mM Sodium Phosphate, 0.5 mg/mL Polysorbate 80, 50 mg/mL sucrose, pH 6,5, diluted 1/200 in medium to correspond to the highest Tocilizumab concentration). All conditions were realised with N=6. Cells were cultivated 2 days at 37° C., 5% CO₂, and the metabolic activity was measured with the WST-1 reagent (Roche).

Primary human chondrocyte culture: The same method and conditions as the one described in example 1 were used. Cells were then harvested with accutase and counted before being inoculated in a 24-well plate at 200 000 cells/well in one mL of complete HAM's F12 supplemented with different concentrations of either Tocilizumab (Roche) in presence or absence of Sprifermin 100 ng/mL. The controls (0 ng/mL Tocilizumab) were realised with cells cultured with or without Sprifermin and in presence of the excipients of the Tocilizumab formulation (see above). All conditions were realised with N=6.

Similar analytic methods as in example 1 were used (for GAG quantification and gene expression analysis).

Results:

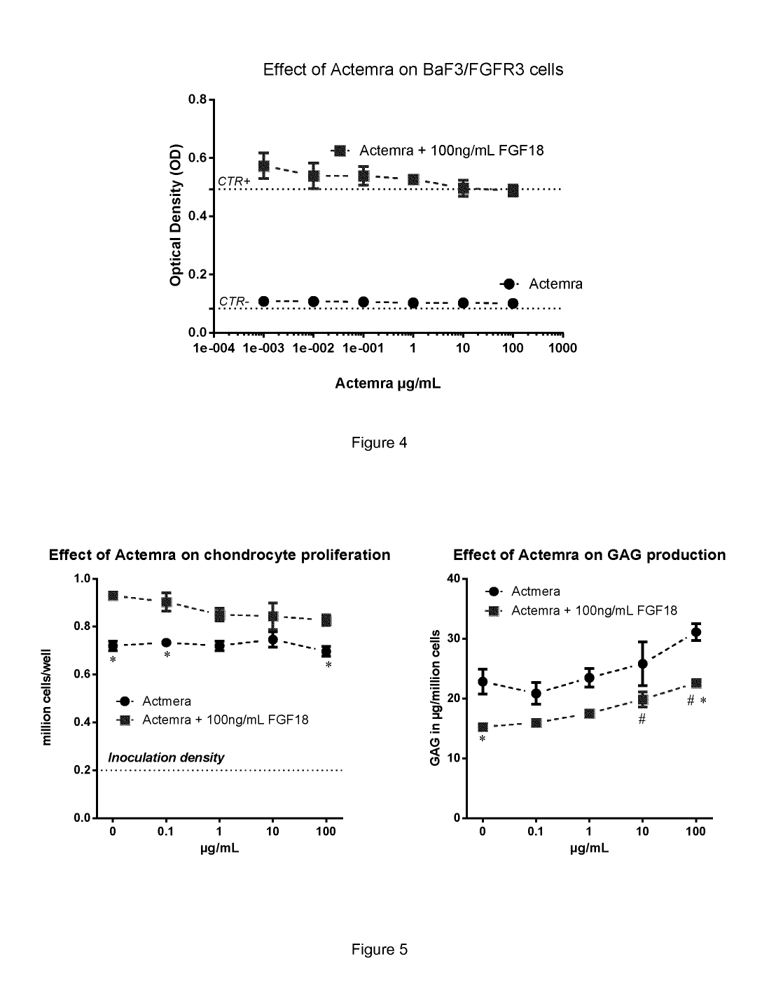

BaF/FGFR3 cell assay (FIG. 4): Tocilizumab had no effect on cell proliferation and did not interfere with Sprifermin. In the absence of Sprifermin, the increasing concentrations of tocilizumab did not influence the cell proliferation and the O.D. remained low. The BaF3/FGFR3 cell proliferation increased in presence of Sprifermin, resulting in an O.D. increasing from about 0.10 (CTR−) to about 0.5 (CTR+). In the presence of Sprifermin, tocilizumab did not show any clear trend. Some small fluctuations of the O.D. were observed but it stayed in the range of the O.D. observed with Sprifermin alone.

Human chondrocytes—Proliferation and GAG production (FIG. 5): As expected, Sprifermin increased chondrocyte proliferation, resulting at the end of the culture in a higher cell concentration (about 0.7 million cells/well in absence of Sprifermin and about 0.9 million cells/well in presence of Sprifermin). This effect was maintained in presence of tocilizumab, which has no effect on proliferation, whatever the concentration, in absence of Sprifermin. The GAG production was slightly decreased when the cells were cultured in continuous presence of Sprifermin. Tocilizumab was found to increased dose-dependently GAG production by human chondrocytes. This effect can be observed in presence or absence of Sprifermin.

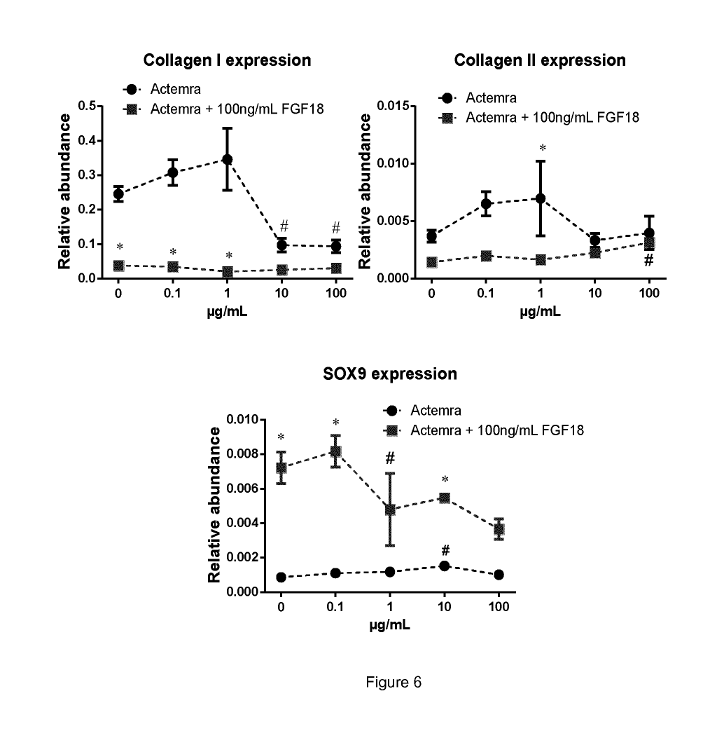

Human chondrocytes—Gene expression (FIG. 6): As expected, sprifermin down regulate Collagen I expression. This effect was not influenced by Tocilizumab. Interestingly, in absence of Sprifermin, Tocilizumab down regulated Collagen I expression only at 10 μg/mL or higher concentrations. The increased of Sox9 expression by Sprifermin was also expected. This effect was decreased by Tocilizumab at concentration above 1 μg/mL, although not inhibited. Finally the effect of tocilizumab on collagen type II was unclear. However, in presence of tocilizumab a significant increase of 2.2 fold of Collagen II expression was observed with 100 μg/mL, compared with sprifermin alone (from 0.0014 to 0.003 relative abundance).

Conclusions: Tocilizumab does not interfere with the bioactivity of Sprifermin. Tocilizumab did not negatively impact the effect of Sprifermin and showed some positive effects in human osteoarthritic chondrocytes: it dose dependently increased GAG production and decreased Collagen I expression. In addition, it increased by a factor 2 Collagen type II expression in chondrocytes cultured in presence of Sprifermin. Although the effect of IL-6R inhibitors on Sox9 expression is unclear, in overall, IL-6 inhibitors seem to be able to increase the anabolic effect of FGF-18.

### Example 3

### Combination of FGF-18 and an Inhibitor of NGF

Method:

BaF3/FGFR3c bioassay: The same method and conditions as the one described in example 1 were used. At the day of the assay 20 000 cells/well were seeded in a 96 well plate in 50 μL of assay medium containing Tanezumab at 0.01, 0.1, 1, 10, 100 or 1 000 nM and containing Sprifermin 100 ng/mL or not. The positive control (CTR +) was realized with cells cultured with Sprifermin 100 ng/mL in absence of Tanezumab. All conditions were realized with N=6. Cells were cultivated 2 days at 37° C., 5% CO₂, and the metabolic activity was measured with the WST-1 reagent (Roche).

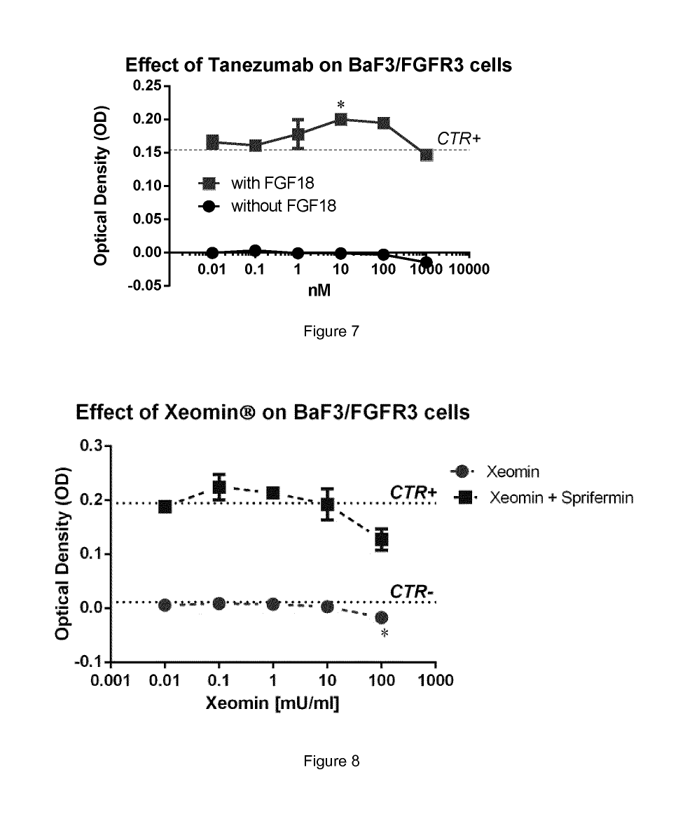

Results (FIG. 7):

Tanezumab had no effect on cell proliferation and did not interfere with Sprifermin. In the absence of Sprifermin, the increasing concentrations of tocilizumab did not influence the cell proliferation and the O.D. remained null. The BaF3/FGFR3 cell proliferation increased in presence of Sprifermin, resulting in an O.D. increasing from about 0 (CTR−) to about 0.15 (CTR+). In the presence of Sprifermin, tocilizumab did not show any particular trend. Some small fluctuations of the O.D. were observed but they stayed in the range of the O.D. observed with Sprifermin alone.

### Example 4

### Combination of FGF-18 and a Botulinum Toxin Compound

Method:

BaF3/FGFR3c bioassay: The same method and conditions as the one described in example 1 were used. At the day of the assay 20 000 cells/well were seeded in a 96 well plate in 50 μL of assay medium containing Xeomin® at 0.01, 0.1, 1, 10 or 100 mU/mL and containing Sprifermin 100 ng/mL or not. As control, cells were also cultivated with Sprifermin 100 ng/mL alone (positive control) or without any compound (negative control). All conditions were realized with N=3. Cells were cultivated 2 days at 37° C., 5% CO₂, and the metabolic activity was measured with the WST-1 reagent (Roche).

Primary bovine chondrocyte culture: The same method and conditions as the one described in example 1 were used. Cells were then harvested with accutase and counted before being inoculated in a 24-well plate at 15 000 cells/well in one mL of complete HAM's F12 supplemented with different concentrations of Xeomin® (1, 10, 100, 1 000 mU/mL) in presence or absence of Sprifermin 100 ng/mL. As control, cells were also cultivated with Sprifermin 100 ng/mL alone (positive control) or without any compound (negative control). All conditions were realized with N=4.

Similar analytic methods as in example 1 were used (for GAG quantification and gene expression analysis).

Results:

BaF/FGFR3 cell assay (FIG. 8): In the absence of Sprifermin, the increasing concentrations of Xeomin® from 0.01 to 10 U/mL did not influence the cell proliferation but at the highest tested concentration (100 U/mL) number of metabolic active cells was significantly reduced. As expected, the BaF3/FGFR3 cell proliferation increased in presence of Sprifermin, resulting in an O.D. increasing from 0.011 (CTR−) to 0.194 (CTR+). In the presence of Sprifermin the same results were observed. Because this decrease in metabolic activity is observed in presence or absence of Sprifermin, it can be concluded that this is a direct effect of Xeomin and not a modulation of Sprifermin bioactivity.

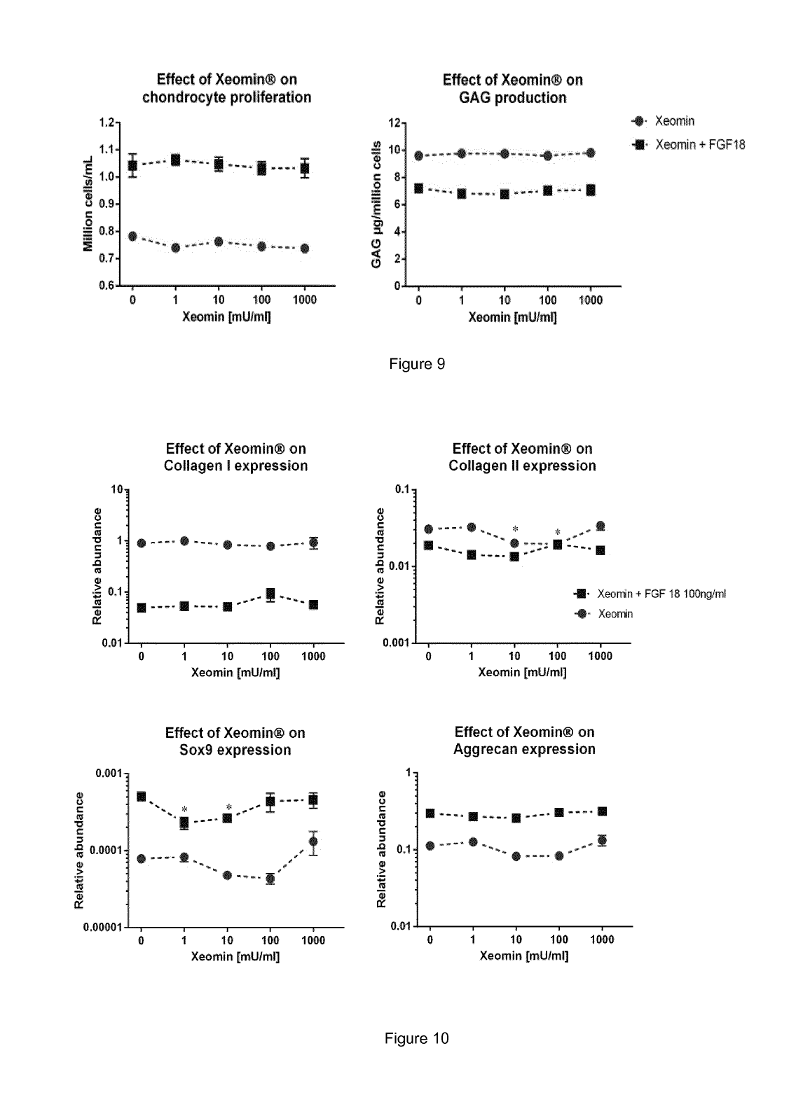

Bovine chondrocytes—Proliferation and GAG production (FIG. 9): As expected, Sprifermin increased chondrocyte proliferation, resulting at the end of the culture in a higher cell concentration (0.78 million cells/well in absence of Sprifermin and 1.04 million cells/well in presence of Sprifermin). This effect was maintained in presence of Xeomin® from 1 to 1 000 mU/mL. Similarly, no effect of Xeomin® on proliferation could be observed in absence of Sprifermin. The GAG production was decreased from 9.6 to 7.2 μg/million cells when cells were cultured in continuous presence of Sprifermin. Both in absence or presence of Sprifermin, Xeomin® from 1 to 1 000 mU/ml was found to have no effect on GAG production.

Bovine chondrocytes—Gene expression (FIG. 10): As expected, Sprifermin in continuous presence down-regulated Collagen I expression (from 0.9 to 0.05) while increasing Sox9 expression (from 7.8×10⁻⁵ to 5.1×10⁻⁴) and aggrecan expression (from 0.11 to 0.3). Sprifermin had also a small effect on Collagen II expression which decreased from 0.031 to 0.018 in presence of Sprifermin. In absence or presence of Sprifermin, Xeomin® from 1 to 1000 mU/mL did not influence Collagen I, II, Sox9 and aggrecan expression.

Conclusions:

As a conclusion, surprisingly at the maximal Xeomin® concentration expected to be found in a human joint (approx. 10 U/mL): 1) no negative effect of Xeomin® could be observed on BaF3/FGFR3 cells and primary chondrocytes (proliferation, phenotype and matrix production were not affected) and 2) no interference of Xeomin® with Sprifermin effects was observed. This was unexpected as both FGF-18 and Botulinum toxin of Type A bind the same receptor, i.e. FGFRIII.

### References

1. Lotz, 2010, Arthritis research therapy, 12:211
2. WO2005080429
3. Boon et al. 2010, PM&R, Vol. 2, 268-276
4. Singh J A et al., 2009, Transl Res;153:205-216
5. Sanga et al., 2013, Pain, 154 :1910-1919
6. Tiseo et al., 2014, Pain, 155 :1245-1252.
7. Ellsworth et al., 2002, Osteoarthritis and Cartilage, 10: 308-320
8. Shimoaka et al., 2002 , JBC 277(9):7493-7500
9. WO2008023063
10. WO2004032849
11. Moore et al., 2005, Osteoarthritis and Cartilage, 13:623-631.
12. WO9816644
13. WO2006063362
14. Custers et al., 2007, Osteoarthritis and Cartilage, 15:1241-1248
15. The Merck manual, 17^{th} edition, 1999
16. ICRS publication: http://www.cartilage.orq/_files/contentmanagement/ICRS_evaluation.pdf, page 13

1-18 (canceled).

19. A composition comprising a combination of at least two active ingredients, wherein one of the active ingredients is an FGF-18 compound and wherein the at least one other active ingredient is selected from the group consisting of an inhibitor of IL-6, an inhibitor of IL-6 receptor, an inhibitor of NGF and a botulinum toxin compound.

20. A method of treating a cartilage disorder comprising administering an FGF-18 compound in combination with at least one additional active ingredient, wherein said at least one additional active ingredient is selected from the group consisting of an inhibitor of IL-6, an inhibitor of IL-6 receptor, an inhibitor of NGF and a botulinum toxin compound.

21. The method according to claim 20, wherein the cartilage disorder is osteoarthritis.

22. The method according to claim 20, wherein the cartilage disorder is cartilage injury.

23. The method according to claim 20, wherein the FGF-18 compound is selected from the group consisting of:
a) a polypeptide comprising the mature form of human FGF-18 mature form, or
b) a polypeptide comprising SEQ ID NO:2.

24. The method according to claim 20, wherein the botulinum toxin compound is botulinum toxin type A.

25. The method according to claim 20, wherein the inhibitor of IL-6 is an anti-IL-6 antibody or an anti-IL-6 nanobody.

26. The method according to claim 20, wherein the inhibitor of IL-6 receptor is an anti-IL-6 receptor antibody or an anti-IL-6 receptor nanobody.

27. The method according to claim 20, wherein the inhibitor of NGF is an anti-NGF antibody or an anti-NGF nanobody.

28. A kit comprising an FGF-18 compound together with instructions for simultaneous or sequential use with at least one additional active ingredient, wherein said at least one additional active ingredient is selected from the group consisting of an inhibitor of IL-6, an inhibitor of IL-6 receptor, an inhibitor of NGF and a botulinum toxin compound.

29. The kit according to claim 28, said kit further comprising an additional active ingredient selected from the group consisting of an inhibitor of IL-6, an inhibitor of IL-6 receptor, an inhibitor of NGF and a botulinum toxin compound.

30. The kit according to claim 29, wherein the FGF-18 compound and the at least one additional active ingredient are part of a pharmaceutical formulation.

31. The kit according to claim 29, wherein the FGF-18 compound and the at least one additional active ingredient are separate pharmaceutical formulations.

32. The kit according to claim 30, wherein the pharmaceutical formulation further comprises at least one excipient.

33. The kit according to claim 31, wherein the pharmaceutical formulations further comprise at least one excipient.

34. The kit according to claim 32, wherein the at least one excipient is selected from the group consisting of a buffer, a surfactant, a salt, an antioxidant, a isotonicity agent, a bulking agent, a stabilizer and any combination thereof.

35. The kit according to claim 33, wherein the at least one excipient is selected from the group consisting of a buffer, a surfactant, a salt, an antioxidant, a isotonicity agent, a bulking agent, a stabilizer and any combination thereof.


SEQUENCE LISTING (14 sequences)

SEQ ID NO: 1 - protein, 207 aa, Homo sapiens
MYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 2 - protein, 170 aa, Artificial
  trFGF-18
MEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTK

SEQ ID NO: 3 - protein, 1295 aa, Clostridium botulinum [sequence omitted]

SEQ ID NO: 4 - protein, 449 aa, Artificial
  heavy chain of CNTO328
EVQLVESGGKLLKPGGSLKLSCAASGFTFSSFAMSWFRQSPEKRLEWVAEISSGGSYTYYPDTVTGRFTISRDNAKNTLYLEMSSLRSEDTAMYYCARGLWGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 212 aa, Artificial
  light chain of CNTO328
IVLIQSPAIMSASPGEKVTMTCSASSSVSYMYWYQQKPGSSPRLLIYDTSNLASGVPVRFSGSGSGTSYSLTISRMEAEDAATYYCQQWSGYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 116 aa, Artificial
  PMP6B6
QVQLVESAGGLVQPGGSLRLSCAASGIIFSINAMGWYRQAPGKRRELVADIMPYGSTEYADSVKGRFTISRDNAKNTVYLQMNSLKPEDTAVYYCHSYDPRGDDYWGQGTQVTVSS

SEQ ID NO: 7 - protein, 448 aa, Artificial
  heavy chain of tocilizumab
  misc_feature (1)..(1): Xaa can be any naturally occurring amino acid
XVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 214 aa, Artificial [sequence omitted]

SEQ ID NO: 9 - protein, 447 aa, Artificial
  heavy chain of tanezumab
QVQLQESGPGLVKPSETLSLTCTVSGFSLIGYDLNWIRQPPGKGLEWIGIIWGDGTTDYNSAVKSRVTISKDTSKNQFSLKLSSVTAADTAVYYCARGGYWYATSYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 214 aa, Artificial
  light chain of tanezumab
DIQMTQSPSSLSASVGDRVTITCRASQSISNNLNWYQQKPGKAPKLLIYYTSRFHSGVPSRFSGSGSGTDFTFTISSLQPEDIATYYCQQEHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 446 aa, Artificial
  heavy chain of fasinumab
QVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIFGVVTNFDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 214 aa, Artificial
  light chain of fasinumab
DIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 449 aa, Artificial
  heavy chain of fulranumab
EVQLVESGGGLVQPGGSLRLSCAASGFTLRSYSMNWVRQAPGKGLEWVSYISRSSHTIFYADSVKGRFTISRDNAKNSLYLQMDSLRDEDTAMYYCARVYSSGWHVSDYFDYWGQGILVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 14 - protein, 214 aa, Artificial
  light chain of fulranumab
AIQLTQSPSSLSASVGDRVTITCRASQGISSALAWYQQKPGKAPKLLIYDASSLESGVPSRFSGSGSGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC